SEQUENCE LISTING (6 sequences)

SEQ ID NO: 1 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 2 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttaccaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 3 - DNA, 8298 bp, synthetic construct
  source 1..8298: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccaacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggccagggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgctacgcgtttaattaaggcgcgcccaattgtgttaatcaataaaccgtttaattcgtttcagttgaactttggtctctgcgtatttctttcttatctagtttccatgctctagagaaacctacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagttcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcctttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgatattttttttttcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccccgcgcgttggccgattcattaatgcag

SEQ ID NO: 4 - DNA, 8717 bp, synthetic construct
  source 1..8717: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgcgtaagctagccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagctcgagaatggccgcggttttttttatcgctgccttgatatacactttccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtaggaagtcttttccacatggcagatgatttgggcagagcgatggagtccttagtatcagtcatgacagatgaagaaggagcagaataaatgttttacaactcctgattcccgcatgcggccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 5 - DNA, 7541 bp, synthetic construct
  source 1..7541: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccgccgccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaattaggcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 7542 bp, synthetic construct
  source 1..7542: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccgccgccaccatgctgtggtgggaggaagtcgaggactgctacgagcgcgaagatgtgcagaaaaagacattcacaaagtgggtaaacgcccagtttagcaagttcggcaagcagcacatcgagaacctattcagcgacctgcaggacggcagaagactgctggacctcctggagggcctgacaggacagaaactgcctaaggagaagggcagcaccagagtgcacgccctgaacaacgtgaacaaagctctacgggttttacagaacaacaatgtggacctggtgaacatcggcagcaccgacatcgtggatggcaaccacaaactgaccctggggcttatctggaacatcatcctgcactggcaggtgaagaacgtgatgaaaaacatcatggccggcctgcagcaaaccaactctgagaagatcctgctgagctgggttaggcagagcacacggaactacccccaggtgaacgtgatcaacttcaccacctcctggtccgacggtttagccctgaacgccctgatccattctcacagaccagacctgttcgactggaacagcgtggtgtgccagcagagtgctacccagcggctggagcacgccttcaacatcgccagataccagctgggcatcgagaagctgctggaccctgaagatgtggacaccacctaccctgacaagaagagcatcctgatgtacatcacatccctatttcaggtcctgccccaacaggtcagcatcgaggccatccaagaggtggagatgctgcctagacctcccaaggtgacaaaagaagaacacttccaactgcaccaccagatgcactacagccagcagatcaccgtgagcctggctcagggatacgagagaacaagcagccctaagcccagattcaagagctacgcctatacacaagccgcctacgtgaccactagcgatcctaccaggtcccctttccccagccagcacctggaggcccctgaggataagtccttcggcagcagcctgatggagagcgaggtgaacctggacaggtatcagaccgctttggaggaagtgctgagctggctgctgagcgccgaggacacattacaggcccagggcgagatcagcaacgacgtcgaggtggtgaaggaccagttccacactcacgagggctacatgatggatctgactgcccaccagggccgggttggaaacatcctgcagctcggaagcaagctgatcggaacaggcaagctgtctgaggatgaggaaaccgaggtgcaggaacagatgaacctgctgaacagcagatgggagtgtctacgggtggccagcatggaaaaacagtctaacctgcacagagtgctgatggacctgcaaaatcagaagctaaaggaactgaatgactggttgacaaaaaccgaggaacggacaagaaagatggaggaagagccgctgggacctgatctggaggacctgaagagacaggtgcagcagcacaaggtgctgcaagaggacctggagcaagaacaggttagagtgaacagcctcacacacatggtggtggtggtggacgagagcagcggcgaccacgccaccgccgcactggaagagcagcttaaagtccttggagacagatgggccaacatttgcagatggaccgaggatagatgggtgctgctgcaggacatcctgctgaagtggcaaagactgacggaagaacagtgcctgttcagcgcctggctgtccgaaaaggaagatgccgtgaacaagatccacaccaccggcttcaaggaccagaacgaaatgctgagcagcttacagaagctggctgtgctgaaagcggacttggagaagaagaagcagagtatgggcaagctgtacagcctgaaacaggacctgctgtcaacgctgaagaacaaaagcgtgacccagaagaccgaggcctggctggacaatttcgcccggtgctgggataatctggtgcagaaactggagaagtctaccgcacagatcagccaggccgtgaccaccacacagcccagcctgacacagacaaccgtgatggaaaccgtgacaaccgtgaccaccagagaacagattctcgtgaagcatgctcaggaagagctgcctcctccacctcctcagaagaaacggcagataacagtagacctggagcggctgcaagagctgcaggaggccacagacgaactggacctgaagctgagacaagctgaagtgatcaagggctcttggcagcctgtgggcgatcttctgatcgacagcctgcaggatcacctggagaaggtgaaagccctcagaggcgagatcgcccctctgaaggaaaacgtgtctcacgtgaacgacctggccagacagctgaccacactgggcattcagctgtctccttacaacctgagcacgctggaggacctgaacacccggtggaagctgctccaggtggccgtggaagatagagtgcgccagctgcacgaagcccacagagatttcggccccgcctctcagcacttcctgtctaccagcgtccagggaccttgggaaagagccatcagccccaacaaggtgccttactacatcaaccacgagacacagacaacctgttgggaccacccaaagatgacagagctgtaccagagcctggctgacctgaacaacgtgcggttcagcgcctacaggaccgccatgaagctgagaagactgcagaaggccctctgtctggacctgctgagcctttccgccgcttgtgacgccctggaccagcacaatctgaaacagaacgaccagcctatggatattctgcaaatcatcaactgcctgaccaccatctacgacagactggagcaggagcacaacaatctggtcaacgtgcccctgtgcgtggatatgtgcctgaattggcttctgaatgtgtacgacaccggacgtaccggccggatcagagtgctgagcttcaaaaccggcatcatatctctgtgcaaggcccatctggaggataagtacagatacctgtttaagcaggtggccagtagcaccggcttctgcgatcagagacggctgggcctgctgctgcatgattctatccagatccccagacaactgggcgaggtggccagcttcggcggctccaacatcgagcccagcgtgcggtcttgcttccaatttgccaataataagcctgagatcgaggctgctctgtttctggactggatgagattggaacctcaaagcatggtgtggctgcctgtgcttcacagagtggctgccgctgagacagctaaacatcaggccaagtgcaacatttgtaaagaatgccctatcatcggctttagataccggagcctgaagcactttaactacgacatctgtcagagctgtttcttcagcggcagagtggccaagggccacaagatgcactaccctatggtggagtactgcacccctacaactagcggcgaagacgtgagagacttcgccaaagtgctgaagaataagttcagaaccaagagatacttcgccaagcacccccggatgggatacctgccagtgcagaccgtgctggaaggcgacaacatggagactgataccatgtgagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2026/0000788 A1
(43) Pub. Date: Jan. 1, 2026

(54) A BIOENGINEERED AAV9 VECTOR CARRYING OPTIMIZED TRANSGENE FOR DUCHENNE MUSCULAR DYSTROPHY GENE THERAPY AND METHOD THEREOF

(71) Applicant: INDIAN INSTITUTE OF TECHNOLOGY KANPUR, KANPUR (IN)

(72) Inventors: JAYANDHARAN GIRIDHARA RAO, Kanpur (IN); MOHAN KUMAR BS, Kanpur (IN); SANYA SHARMA, Kanpur (IN); NARENDRA KUMAR, Kanpur (IN)

(21) Appl. No.: 19/240,179

(22) Filed: Jun. 17, 2025

(30) Foreign Application Priority Data
Jun. 18, 2024 (IN) 202411046835

(51) Int. Cl. A61K 48/00; A61K 38/17; C07K 14/47; C12N 15/86

(52) U.S. Cl. CPC A61K 48/005; A61K 38/1709; A61K 48/0075; C07K 14/4708; C12N 15/86; C12N 2750/14143; C12N 2750/14152

(57) ABSTRACT

The invention, in general, relates to the field of Adeno-associated virus (AAV). More particularly, the present invention relates to bioengineered AAV9 vector carrying optimised transgene for Duchenne muscular dystrophy gene therapy. The present invention particularly provides an engineered AAV9 vector containing a microdystrophin therapeutic gene optimized for codon usage, under the control of a ubiquitous promoter and a Kozak sequence, aimed at gene therapy for Duchenne muscular dystrophy. The improved AAV9 vector amplifies the therapeutic efficacy of DMD gene therapy.

### EARLIEST PRIORITY DATE

This application claims priority from a Provisional patent application filed in India having patent application No. 202411046835, filed on 18 Jun. 2024 and titled “A BIOENGINEERED AAV9 VECTOR CARRYING OPTIMIZED TRANSGENE FOR DUCHENNE MUSCULAR DYSTROPHY GENE THERAPY AND METHOD THEREOF”.

### FIELD OF THE INVENTION

The invention, in general, relates to the field of Adeno-associated virus (AAV). More particularly, the present invention relates to bioengineered AAV9 vector carrying optimised transgene for Duchenne muscular dystrophy gene therapy.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Duchenne muscular dystrophy (DMD) is a severe neuromuscular disorder in humans (1:3500 male births) caused by mutations in the DMD gene and the subsequent loss of dystrophin protein. This leads to a compromised dystrophin glycoprotein complex (DGC), crucial in connecting the cytoskeleton to the muscle fibre cell membrane. The phenotypic manifestations are severe, ranging from muscle fibre necrosis, inflammation which manifests as cardiomyopathy, cognitive impairment, and ambulatory issues due to overall lack of muscle functioning. Many DMD patients eventually succumb to cardiac or respiratory failure by the second decade of their life.

The current standard of care for DMD patients includes administration of corticosteroids such as deflazacort and prednisone, that has limited efficacy in halting the disease progression and prolong ambulation in treated patients. Newer therapeutic approaches through antisense oligonucleotide (ASO)-mediated exon skipping, stop codon readthrough and gene replacement are promising. Gene replacement with Adeno-associated viral vectors (AAV) has multiple advantages such as broader tissue tropism, non-pathogenicity, and providing stable therapeutic gene expression. Multiple serotypes of recombinant AAV have been extensively used for genetic therapies. Of these, AAV9 serotype has a greater transduction in skeletal and cardiac muscle cells and hence an ideal vector system for targeting muscle diseases such as DMD. Due to the maximum packaging limit of ˜4.4 kb in AAV vectors, micro dystrophin—a truncated product of the dystrophin gene is widely used for gene therapy of DMD.

In preclinical models, AAV-micro dystrophin demonstrated promising transduction to striated muscles, reduced cardiac fibrosis, and improved cardiac performance. In clinical trials, such therapies show a modest functional improvement in DMD patients even though an extremely higher dose of 10¹³-10¹⁴ vector genomes (vgs) of AAV is required. However, such a higher dose has resulted in inflammatory myopathy in dog pups and liver toxicity in rhesus macaques attributed to severe immune response. Incidentally, a high dosage DMD clinical trial reported death of a patient. In another clinical trial, evidence for complement activation found in a patient after high dose AAV injection (2×10¹⁴ vgs/kg). Multiple lines of evidence suggest that immune response to AAV vectors is dose dependent. Thus, to address this crucial issue of high vector doses and concomitant immune response, a precise selection of appropriate promoter, the transgene and capsid combination is required to enhance therapeutic outcomes.

Hence, there is an urgent need to provide an efficient bioengineered AAV9 vector which will enhance the therapeutic potential post gene therapy.

### OBJECTIVE OF THE INVENTION

The primary object of the present invention is to overcome the drawbacks associated with prior art.

Another object of the present invention is to provide an optimised Adeno-associated virus (AAV) based gene therapy for an improved dystrophin gene function.

Another object of the present invention is to provide a modified AAV9 capsid at their rate limiting post translational modification sites to increase their transduction efficiency thereby enhancing the therapeutic outcome post gene therapy.

Another object of the present invention is to provide a bioengineered AAV9 vector carrying a codon optimised microdystrophin therapeutic gene regulated by ubiquitous promoter and a Kozak sequence for Duchenne muscular dystrophy gene therapy. This optimal AAV9 vector will enhance the therapeutic potential post gene therapy.

Another object of the present invention is to provide a combination of rational engineering of AAV9 capsids strategically modified at the rate limiting post translational modification sites (K51Q, N57Q), combined with promoter selection (muscle specific MHCK7 vs. ubiquitous CAG) and codon optimization of the micro dystrophin (uDys) transgene to enhance the functionality of AAV9 vectors.

Another object of the present invention is to provide the bioengineered Adeno-associated virus (AAV) vectors for delivering therapeutic genes due to their safety profile and efficiency in transducing muscle tissue.

Another object of the present invention is to provide an optimal AAV9 vector to enhance the therapeutic potential post gene therapy.

### SUMMARY OF THE INVENTION

The Invention provides a Bioengineered AAV9 Vector Comprising Optimized Transgene For Duchenne Muscular Dystrophy Gene Therapy, comprising a novel mutations present at the post-translational site/s of the vector;
said the gene sequence comprises optimised microdystrophin transgene with ubiquitous promoter comprising CAG promoter and Kozak sequence upstream of the transgene.

The vector comprises AAV9 and its mutant vectors comprising K51Q, N57Q, comprising sequence Id no. 2 and 3 respectively. In an embodiment, the ubiquitous promoter containing micro-dystrophin vector is constructed by the steps of:

constructed the plasmid vector by sub-cloning the ΔR4-23/AC micro-dystrophin gene (μDys) from the donor plasmid pLV-I-μDys to the recipient plasmid pssAAV-CAG-eGFP, wherein the recipient plasmid comprises a hybrid promoter/enhancer sequence (CAG) with the combination of a cytomegalovirus (CMV) immediate early enhancer sequence and chicken β actin (CBA) promoter;
inserting a consensus ribosome binding site sequence comprising Kozak sequence being inserted downstream to the CAG promoter, such that the micro-dystrophin gene is placed downstream to the CAG-Kozak sequence in the final construct designated as pssAAV-CAG-Kozak-micro-dystrophin (CAG-Kozak-μDys) comprising sequence Id 5, to ensure the ubiquitous and greater translational efficacy of dystrophin.
a. In an embodiment, the next generation AAV9 vectors are constructed by engineering the capsids at PTM sites and packaging them with pssAAV-CAG-Kozak-μDys transgene construct to generate AAV9WT-CAG-Kozak-μDys (AAV9WT-μDys), AAV9K51Q-CAG-Kozak-μDys (AAV9K51Q-μDys) and AAV9N57Q-CAG-Kozak-μDys (AAV9N57Q-μDys).
b. The optimal AAV9 vector was generated by packaging the PTM modified AAV9K51Q vector with codon-optimised pssAAV-CAG-Kozak-CouDys transgene to generate AAV9K51Q-CAG-Kozak-CouDys (AAV9K51Q-CouDys).
c. In an embodiment, the method of preparing a Bioengineered AAV9 Vector Carrying Optimized Transgene for Duchenne Muscular Dystrophy Gene Therapy comprises the steps of:
selection of a hybrid chicken β-actin promoter and cytomegalovirus early enhancer (CAG) for expression of dystrophin;
incorporating a consensus ribosome binding site sequence or Kozak sequence, after the CAG promoter upstream of the transgene sequence to ensure increased and robust expression of the therapeutic protein;
performing capsid engineering at PTM sites to generate AAV9 mutant vectors with increased transduction efficiency;
performing codon optimization of the μDys (CouDys) transgene for optimal expression in human skeletal muscles.

The method wherein codon optimization of the μDys (CouDys) transgene is performed by the steps:
a) constructing pssAAV-CAG-Kozak-CouDys (CAG-Koz-CouDys) comprising sequence Id 6, by incorporating the chemically synthesized CouDys sequence within the pssAAV-CAG-backbone plasmid;
b) packaging CAG-Koz-CouDys construct in AAV9K51Q capsid to obtain AAV9K51Q-CAG-Kozak-CouDys vectors (AAV9K51Q-CouDys).

AAV9 based vector is injected by intramuscular or systemic administration at the dose range of 1×10¹⁰ vgs to ×10¹⁴ vgs.

### DETAILED DESCRIPTION OF THE DRAWINGS

To further clarify the advantages and features of the present invention, a more particular description of the invention will be rendered by reference to specific embodiments thereof, which are illustrated in the appended drawings. It is appreciated that these drawings depict only typical embodiments of the invention and are therefore not to be considered limiting in their scope. The invention will be described and explained with additional specificity and detail with the accompanying drawings in which:

Figure A: Illustrates a graphical abstract of the present invention to show that the combination of engineered AAV9 vectors optimized for their promoter, codon utilization from the transgene and coupled with improved AAV9 mutant capsids significantly contributes to efficacy of intramuscular gene therapy in DMD mice in vivo.

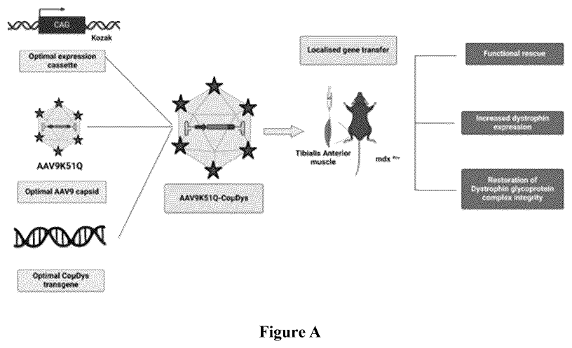

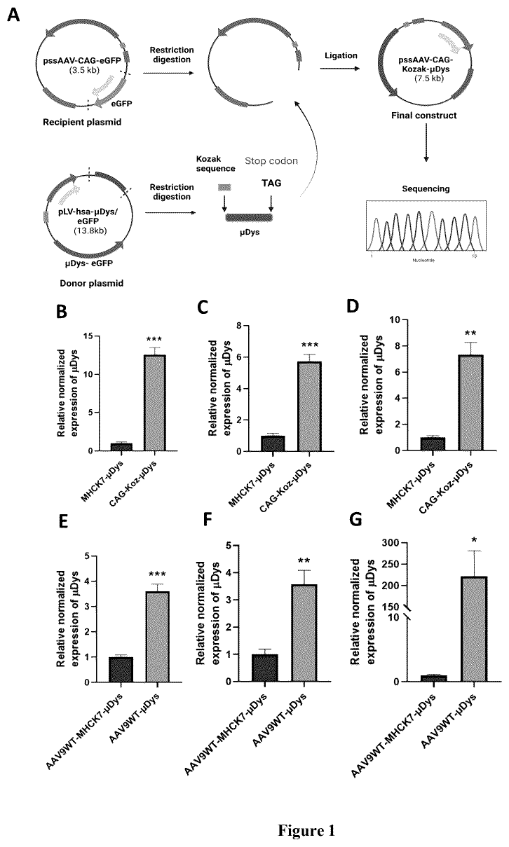

FIG. 1. Designing of micro dystrophin constructs and its validation in vitro. (A) The plasmid pssAAV-CAG-Kozak-μDys was cloned from pLV-hsa-μDys to pAAV-CAG-eGFP with an additional Kozak sequence upstream of μDys. Transfection data from micro-dystrophin vectors in HeLa (B), C2C12 (C) and H9C2 (D) cells is depicted. Similarly, the viral vectors were packaged in AAV9WT capsid, and transduction was performed in HeLa (E), C2C12 (E) and H9C2 (F) cells. A quantitative (q) PCR was performed to detect dystrophin expression. The expression levels were compared to the cell control group and normalized to 18S rRNA expression. The data is represented as mean±S.D. * represents statistical comparisons with respect to the cell control group. *-p<0.05, **-p<0.01, ***-p<0.001. The data is representative of one independent experiment (total, n=2) with three technical replicates per group. qPCR data was analyzed by the Biorad CFX Manager 3.1. Image created using Biorender.com.

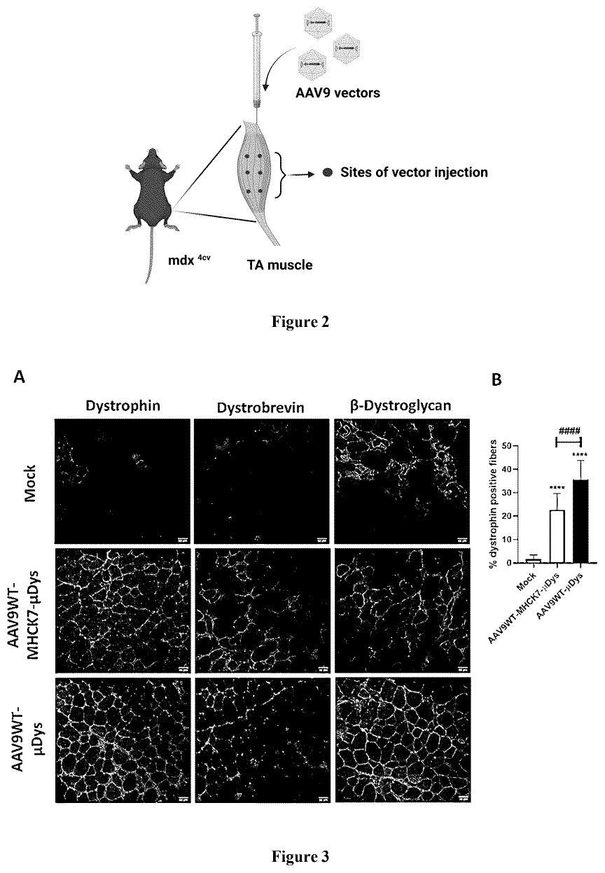

FIG. 2. Intramuscular administration of AAV9 vector in TA muscle of DMD mice. For vector administration, 6-10 weeks old mdx mice were anesthetized and the TA muscle was exposed by an incision. Vectors were administered using a Hamilton syringe and the incision was sutured following the administration. Image created using Biorender.com.

FIG. 3. AAV9 vectors containing a ubiquitous promoter has shown enhanced dystrophin expression. Immunohistochemistry of TA muscle of vector treated mice was performed and representative images of dystrophin-glycoprotein complex (DGC) protein expression 33 weeks after AAV9WT and AAV9MHCK7 vector administration is shown (A). Quantification of the percentage of dystrophin positive fibers was performed as described in the methods (B). Representative images were captured at a magnification of 20× with Advanced High Sensitive Spectral Confocal and Multiphoton Microscope LSM780NLO, CarlZeiss GmbH, Wein, Austria. Scale bar is 50 μm. The quantification of dystrophin-positive fibers was done from images (n≥30/group from n=4 animals/group) captured in an inverted microscope (Dmi8, Leica Microsystems, Germany) in 20× magnification. To quantify the number of total muscle fibers in a particular field, TA muscle sections (n=3 animals) were stained with wheat germ agglutinin and imaged in an inverted microscope (Dmi8, Leica Microsystems, Germany) in 20× magnification. These wheat germ agglutinin stained TA section images (n=50) were used to quantify the number of total muscle fibers (˜74) (data not shown). The number of dystrophin positive fibers and total fiber number was manually counted and marked in the images using ImageJ software and their percentage is denoted in B. The data are represented as mean±S.D. * represents statistical comparison of AAV9WT-MHCK7-μDys and AAV9WT-μDys with respect to the mock and #refers to statistical comparison of AAV9WT-μDys with respect to AAV9WT-MHCK7-μDys experimental group. ****/####-p<0.0001. For statistical analysis, a one-way ANOVA was performed with GraphPad Prism 8.0.2 software.

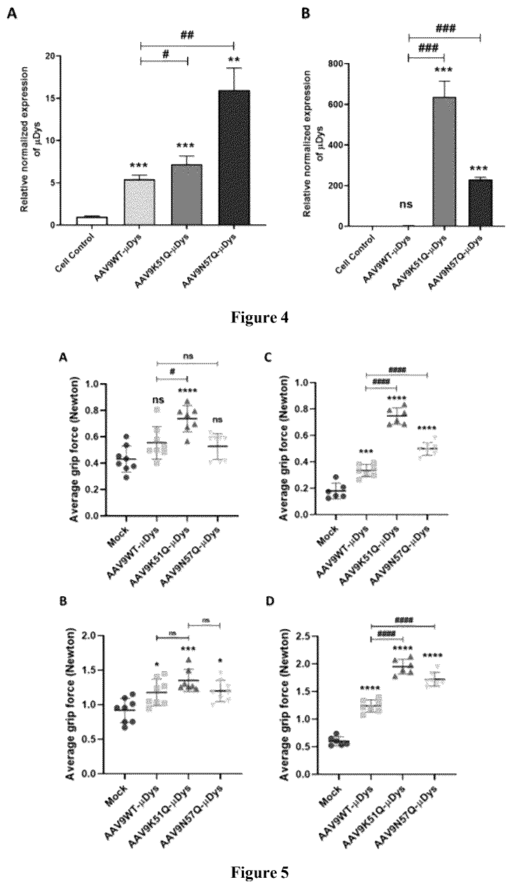

FIG. 4. Rationally engineered AAV9 vectors demonstrates enhanced transduction efficiency. Transduction assay was performed to assess the efficiency of engineered AAV9 vectors packaged with CAG-Kozak-microdystrophin. Infection of HeLa (A) and C2C12 cells (B) was performed at an MOI of 1×10⁵ vgs/cell. Quantitative (q) PCR was performed to detect micro-dystrophin expression. The expression levels were compared to the cell control group and data normalized to 18S rRNA levels. The data obtained is represented as mean±S.D. * represents statistical comparisons with respect to the cell control and #refers to statistical comparison with respect AAV9WT-μDys. ns-non-significant, #-p≤0.05, **/##-p<0.01, ***/###-p≤0.001. The depicted data is from an independent experiment with three technical replicates per group. qPCR data was analyzed by the Biorad CFX Manager 3.1.

FIG. 5. Muscle grip strength is enhanced in mdx mice treated with engineered AAV9 vectors. Muscle grip strength of vector treated hind limbs (A) and all four limbs (B) is represented, at 12 weeks post gene therapy for mock (n=8), AAV9WT-μDys (n=8), AAV9K51Q-μDys (n=7) and AAV9N57Q-μDys (n=8). Follow-up of 18 weeks post vector administration for grip strength of hind limbs (C) and four limbs (D) in mock (n=6), AAV9WT-μDys (n=7), AAV9K51Q-μDys (n=6) and AAV9N57Q-μDys (n=7). At 12 weeks post vector administration one animal from each group was withdrawn for IHC analysis and one animal from mock group died. Each data point represents the average value of 5 readings measured per mice. The data are represented as mean±S.D. * represents statistical comparisons with respect to the Mock and #refers to statistical comparison with respect AAV9WT-μDys. ns-non-significant, */#-p≤0.05, ***-p<0.001, ****/####-p<0.0001 Statistical tests were performed by one way ANOVA executed with GraphPad Prism 8.0.2 software.

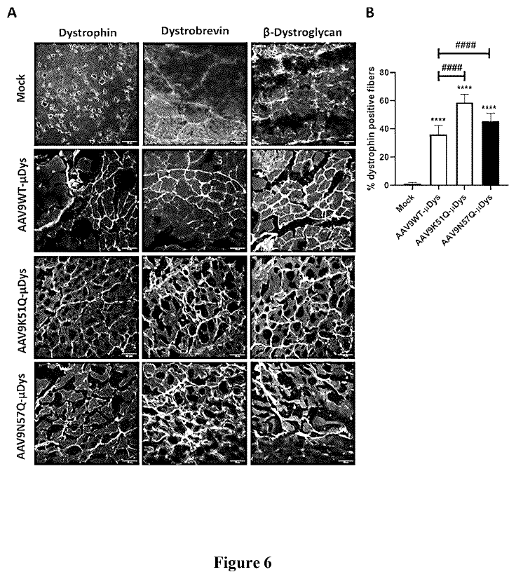

FIG. 6. Localization of DGC proteins in TA muscle sections of mice administered with PTM modified AAV9 vectors. Representative immunofluorescence images for the expression of dystrophin-glycoprotein complex (DGC) proteins in TA muscle of mdx mice, 24-weeks after vector administration (A). Quantification of dystrophin positive fibers in treated mice (B). The sections were imaged with the Carl Zeiss LSM780NLO confocal microscope with a 40× objective (Scale bar: 50 μm). The quantification of dystrophin-positive fibers was done from images (n≥15/group) captured in an inverted microscope (Dmi8, Leica Microsystems, Germany) in 20× magnification. For each group, n=3 animals and n=3 sections per muscle were chosen for imaging analysis. The TA muscle sections (n=3 animals) were stained with wheat germ agglutinin to label the sarcolemma and imaged in an inverted microscope (Dmi8, Leica Microsystems, Germany) in 20× magnification. These wheat germ agglutinin stained images (n=55 from four different animals) were used to quantify the total muscle fiber number (˜78) (data not shown). The number of dystrophin positive fibers and total fiber number was manually counted and marked in the images using ImageJ software and their percentage is denoted in B. The data obtained are represented as mean±S.D. * represents statistical comparison of AAV9WT-μDys, AAV9K51Q-μDys, AAV9N57Q-μDys with respect to the Mock and #refers to statistical comparison of AAV9K51Q-μDys, AAV9N57Q-μDys with respect to AAV9WT-μDys. ****/####-p<0.0001. For statistical comparison, a one way ANOVA was performed (GraphPad Prism 8.0.2 software).

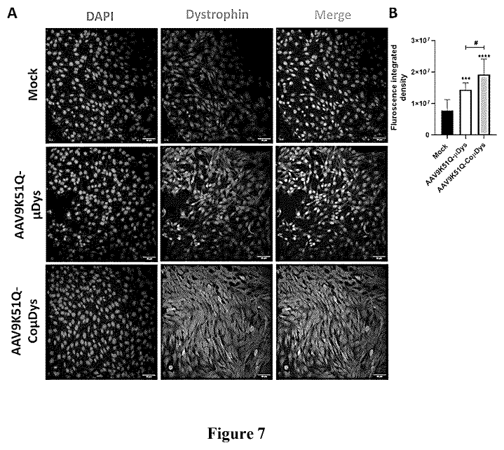

FIG. 7. Validation of Codon optimised microdystrophin transgene in vitro. Transduction of AAV9K51Q-μDys and AAV9K51Q-CouDys vectors in C2C12 cells (A), at an MOI: 1×10⁵ and its quantification (B) is shown. Representative images were obtained in confocal microscope (AXR Nikon) with a 40× objective (Scale bar: 50 μm). Semi-quantification was performed from n≥8 images per group obtained in ZOE-Fluorescent cell imager. Scale bar is 50 μm. The data obtained are represented as mean±S.D. * represents statistical comparisons with respect to the cell control and #refers to statistical comparison with respect AAV9K51Q-μDys. #-p<0.05, ***-p<0.001, ****-p<0.0001. Statistical tests were performed by one way ANOVA with GraphPad Prism 8.0.2 software.

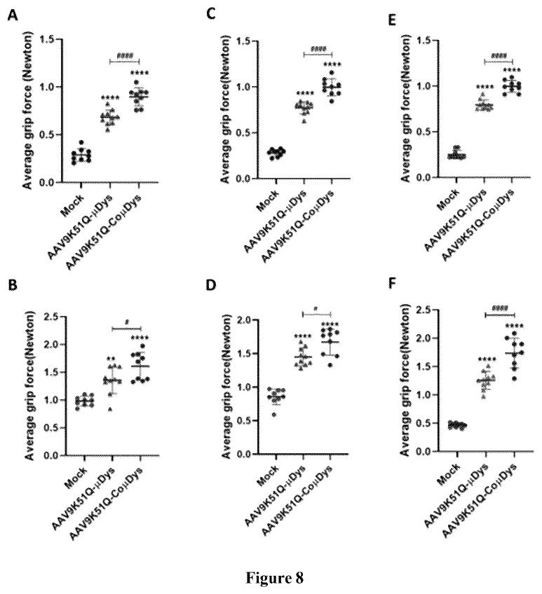

FIG. 8. Muscle strength of mice administered with engineered AAV9 capsid and codon optimized vector. Grip strength was obtained as described in the methods section. Data obtained after 8 weeks of gene transfer for hind limbs, (A), all four limbs (B) followed by 12 weeks follow up for hind limbs (C) and four limbs (D) are depicted. A further long-term follow-up of 16 weeks for hind limbs (E) and four limbs (F) of mice treated with AAV9K51Q-μDys (n=10) and AAV9K51Q-CouDys (n=9) vectors is significantly higher than mock treated group (n=10). Each data point represents the average value of 5 readings measured per mice. The data obtained are represented as mean±S.D. * represents statistical comparisons with respect to the mock and #refers to statistical comparison with respect AAV9K51Q-μDys. #-p<0.05, ##-p<0.01, ****/####-p≤0.0001. Average of 5 technical replicates per mice in grip strength analysis. Statistical tests were performed one way ANOVA executed with GraphPad Prism 8.0.2 software.

FIG. 9. Immunofluorescence staining exhibits increased levels of DGC proteins in TA muscles of optimized AAV9 vector administered animals. The expression of dystrophin-glycoprotein complex (DGC) proteins in TA muscle of mdx mice, 17 weeks after administration of AAV9K51Q-μDys, AAV9K51Q-CouDys vectors (A) and quantitative analysis of dystrophin positive fibers (B) are shown. Images are representative for each group. The sections were imaged with the confocal microscope (AXR Nikon) with a 40× objective (Scale bar: 50 μm). The whole section images of the same dystrophin-stained TA muscles are represented in FIG. 10 (Magnification 2×). The quantification of dystrophin-positive fibers was done from images (n≥15/group from n=3 section/muscle from n=3 animals/group) captured in an inverted microscope (Dmi8, Leica Microsystems, Germany) in 20× magnification. The TA muscle sections (n=3 animals) was stained with wheat germ agglutinin to label the sarcolemma and imaged in an inverted microscope (Dmi8, Leica Microsystems, Germany) in 20× magnification. These wheat germ agglutinin stained images (n=17 from three different animals) were used to quantify the total muscle fiber number (˜71) (data not shown). The number of dystrophin positive fibers and total fiber number was manually counted and marked in the images using ImageJ software and their percentage is denoted in B. The data are represented as mean±S.D. * represents statistical comparison of AAV9K51Q-μDys and AAV9K51Q-CouDys with respect to the Mock and #refers to statistical comparison of AAV9K51Q-CouDys with respect to AAV9K51Q-μDys. ###-p<0.001 ****-p<0.0001. For statistical comparison, a one way ANOVA was performed (GraphPad Prism 8.0.2 software).

FIG. 10. Uniform expression of dystrophin in vector treated DMD mice. Whole section immunofluorescence images of dystrophin stained TA muscles of mdx mice, 17 weeks after Mock or AAV9K51Q vectors administration. The sections were imaged with the confocal microscope (AXR Nikon) with a 2× objective (Scale bar: 1000 μm). The magnified dystrophin stained TA muscle sections imaged in 40× magnification are represented in FIG. 9A.

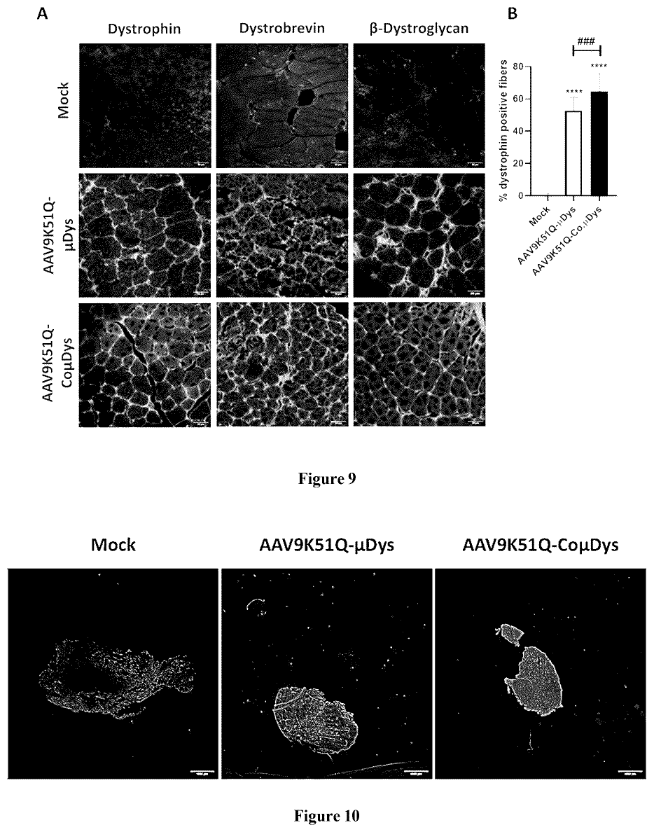

FIG. 11. Collagen deposition in vector administrated DMD mice. Masson trichrome (MT) staining of TA muscle sections from mock or vector treated mice (after 17 weeks) was performed. Yellow arrows refer to collagen deposition (A). The top panel, A (i-iii) represents the whole TA muscle section images (Magnification: 4×) from Mock or AAV9K51Q vector-treated mice (Scale bar: 500 μm). The magnified images (20× magnification) of the same MT-stained TA muscle sections are depicted in the bottom panel, A (iv-vi) (Scale bar: 50 μm). Images were captured in an inverted light microscope (Dmi8, Leica Microsystems, Germany). Collagen deposited areas in MT images were quantified using Image J analysis as described in methods section (B). Three tissues per group were analyzed (n≥10 images/group). The data are represented as mean±S.D. * represents statistical comparison of AAV9K51Q-μDys and AAV9K51Q-CouDys with respect to the mock and #refers to the statistical comparison of AAV9K51Q-CouDys with respect to AAV9K51Q-μDys experimental group. ****/####-p<0.0001. For statistical analysis, a one-way ANOVA was performed with GraphPad Prism 8.0.2 software.

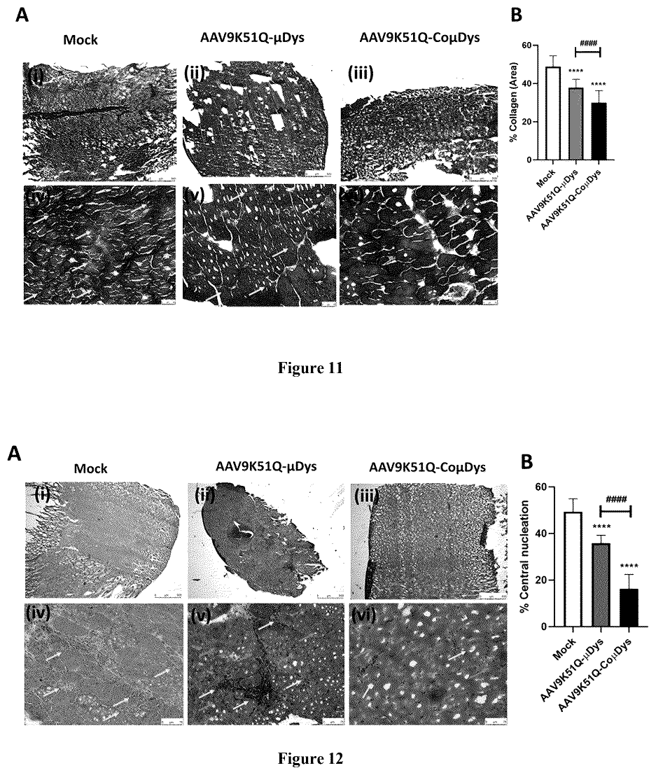

FIG. 12. Morphological analysis of vector administrated DMD mice. Hematoxylin and Eosin (H & E) staining of TA muscle sections from vector treated mice (after 17 weeks) was performed. Green arrows refer to centrally located nucleus in myofibers and yellow arrow denotes the fibrotic areas (A). The top panel, A (i-iii) represents the whole TA muscle section images (Magnification: 4×) from Mock or AAV9K51Q vector-treated mice (Scale bar: 500 μm). The magnified images (20× magnification) of the same H&E-stained TA muscle sections are depicted in the bottom panel (iv-vi). (Scale bar: 75 μm). Images were captured in an inverted microscope (Dmi8, Leica Microsystems, Germany). Quantification of centrally nucleated myofibers in H & E images was performed using Image J analysis as described in methods section (B). H & E images (n≥15 images/group) from n=2-3 tissues per group were used for quantification analysis. The data are represented as mean±S.D. * represents statistical comparison of AAV9K51Q-μDys and AAV9K51Q-CouDys with respect to the mock and #refers to statistical comparison of AAV9K51Q-CouDys with respect to AAV9K51Q-μDys experimental group. ****/####-p<0.0001. For statistical analysis, a one-way ANOVA was executed with GraphPad Prism 8.0.2 software.

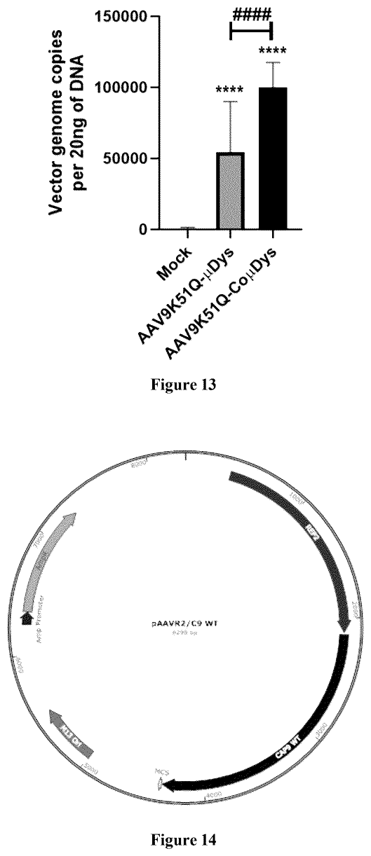

FIG. 13. Vector genome quantification of TA muscles from vector administrated DMD mice. Vector copy numbers in the TA muscles were analysed using quantitative PCR. Briefly, total DNA (n=4 animals/group) was isolated and 20 ng of DNA was amplified using ITR specific primer pair. The quantification was performed using suitable plasmid standards. The data is represented as mean±S.D. * represents the statistical comparison of AAV9K51Q-μDys and AAV9K51Q-CouDys with respect to the mock and #refers to statistical comparison of AAV9K51Q-CouDys with respect to AAV9K51Q-μDys experimental group. ****/####-p<0.0001. For statistical analysis, a one-way ANOVA was executed with GraphPad Prism 8.0.2 software.

FIG. 14 illustrates plasmid map of pAAVR2/C9 WT (Sequence Id 1), with Rep2-Nucleotides 378 to 2117 and Cap9WT-Nucleotides 2134 to 4344

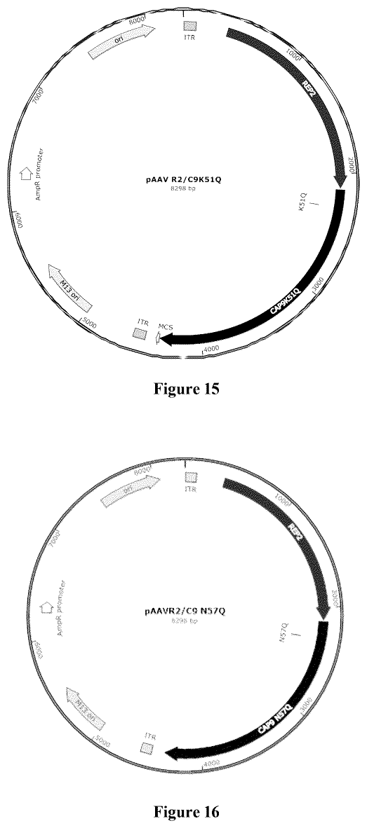

FIG. 15 illustrates Plasmid map of pAAVR2/C9 K51Q where REP2-Nucleotides 378 to 2117 (Sequence Id 2), CAP9 K51Q-Nucleotides 2134 to 4344 while pAAV9/C9 K51Q contains CAA at the nucleotides from 2284 to 2286

FIG. 16 illustrates plasmid map of pAAVR2/C9 N57Q where, REP2-Nucleotides 378 to 2117 (Sequence Id 3), CAP9 K57Q-Nucleotides 2134 to 4344 and pAAV9/C9 N57Q contains CAG at the nucleotides from 2302 to 2304

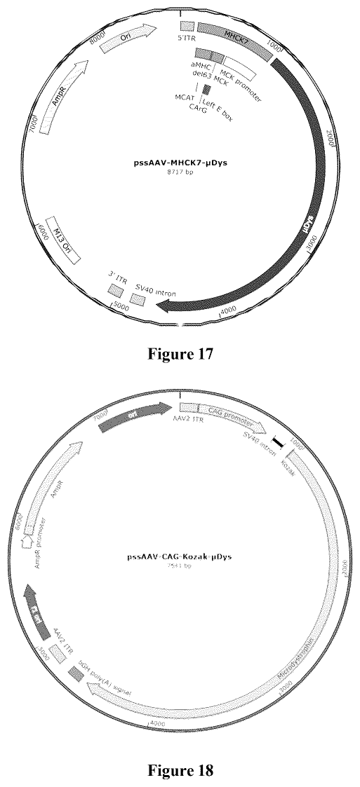

FIG. 17 illustrates plasmid map of pssAAV-MHCK7-μDys where MHCK7 promoter-Nucleotides 175 to 964 (Sequence Id 4), μDys-Nucleotides 1009-4599

FIG. 18 illustrates plasmid map of pssAAV-CAG-Kozak-μDys where (′AG promoter-Nucleotides 148 to 714 (Sequence Id 5), Kozak-Nucleotides 952-960, μDys-Nucleotides 961-4550

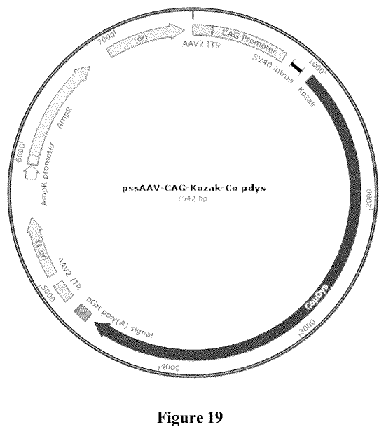

FIG. 19 illustrates plasmid map of pssAAV-CAG-Kozak-CouDys where CAG promoter-Nucleotides are from 148 to 714 (Sequence Id 6), Kozak-Nucleotides are from 952 to 960, CouDys-Nucleotides 961 to 4551

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Incorporation by Reference: A Sequence Listing XML file, named Sequence Listing_202411046835.xml, created on 17 Jun. 2025, and having a size of 55,296 bytes (approximately 54 KB), is hereby incorporated by reference pursuant to 37 CFR 1.835(a)(2). The substitute specification submitted herewith contains no new matter relative to the originally filed specification.

The present invention provides a modified AAV9 vector at their post translational sites carrying a novel human codon optimised microdystrophin transgene with CAG promoter and Kozak sequence upstream of the transgene.

In an aspect, the bioengineered AAV9 vector carries a codon optimised microdystrophin therapeutic gene regulated by ubiquitous promoter and a Kozak sequence for Duchenne muscular dystrophy gene therapy. This optimal AAV9 vector will enhance the therapeutic potential post gene therapy.

In an embodiment, the invention employs three overlapping strategies to develop an optimized AAV9 vector for DMD gene therapy. This includes selection of a hybrid chicken β-actin promoter and cytomegalovirus early enhancer (CAG) for widespread and robust expression of dystrophin. Additionally, a consensus ribosome binding site sequence, known as the Kozak sequence, was incorporated after the CAG promoter upstream of the transgene sequence to ensure increased and robust expression of the therapeutic protein. Taking advantage of the superior transduction capability of the AAV9 serotype in muscle tissues, capsid engineering was performed at PTM sites to generate AAV9 mutant vectors with increased transduction efficiency. Furthermore, to maximize transgene expression and overcome limitations such as codon bias and the availability of cognate tRNAs affecting translational efficiency, codon optimization of the μDys (CouDys) transgene was carried out for optimal expression in human skeletal muscles. The Inventors have assessed the proof-of-concept efficacy of these triple engineered vectors in multiple cell line models in vitro and by local intramuscular administration in a mouse model (mdx) of DMD.

### The Invention is Further Described with the Help of Non-Limiting Examples and their Results Design and Evaluation of a Ubiquitous Promoter Containing Micro-Dystrophin Vector

The Inventors constructed the plasmid vector by sub-cloning the ΔR4-23/AC micro-dystrophin gene (μDys) from the donor plasmid pLV-I-μDys to the recipient plasmid pssAAV-CAG-eGFP (GenScript, Piscataway, NJ, US). The recipient plasmid contains a hybrid promoter/enhancer sequence i.e, the combination of a cytomegalovirus (CMV) immediate early enhancer sequence and chicken β actin (CBA) promoter known as CAG promoter which has proven ubiquitous expression in various cell lines and cell types. To increase the translation efficiency of the transgene, a consensus ribosome binding site sequence termed as Kozak (GCCGCCACC) was inserted downstream to the CAG promoter. Thus, the micro-dystrophin gene was placed downstream to the CAG-Kozak sequence in the final construct designated as pssAAV-CAG-Kozak-micro-dystrophin (CAG-Kozak-μDys) to ensure the ubiquitous and greater translational efficacy of dystrophin (FIG. 1A). The Inventors then evaluated the efficacy of this plasmid, in vitro in different muscle cell lines like murine myoblast (C2C12) and rat cardiomyocytes (H9C2) and non-myogenic cell line like human cervical carcinoma (HeLa) cells. The transgene pssAAV-MHCK7-μDys (MHCK7-μDys) (a kind gift from Dr. Jeffrey Chamberlain, University of Washington) was used as a control. Following transfection, the comparison of steady-state mRNA levels of ΔR4-23/AC micro-dystrophin revealed that the CAG-Kozak construct demonstrated a 12-fold (p<0.001) increased expression compared to the MHCK7 promoter in HeLa cells (FIG. 1B). Similarly, a significantly higher micro-dystrophin expression (5-fold; p<0.001 and 7-fold; p<0.01) was observed with the CAG-Kozak driven construct in C2C12 cells and H9C2 cells respectively, when compared to the muscle-specific promoter (FIGS. 1C and 1D). Further, both the constructs of micro-dystrophin were packaged in AAV9 wild type (AAV9WT) capsid due to its tropism towards muscle. Transduction of AAV9WT-CAG-Koz-μDys (AAV9WT-μDys) and AAV9WT-MHCK7-μDys vectors revealed a three-fold difference between CAG-Kozak and MHCK7 driven micro-dystrophin expression levels in both HeLa and C2C12 cells (FIGS. 1E and 1F). Similarly, in vitro analysis in H9C2 cells revealed ˜221-fold (p<0.05) increased expression of μDys in AAV9WT-μDys infected cells when compared to cells infected with AAV9WT-MHCK7-μDys vectors (FIG. 1G).

a. The Inventors further evaluated the efficiency of CAG-Kozak and MHCK7 driven μDys constructs packaged in AAV9WT vector in a mice model of DMD (mdx, n=8 per group). An intramuscular administration of AAV9WT-μDys and AAV9WT-MHCK7-μDys vectors in the Tibialis anterior (TA) was performed at a dose of 3.4×10¹¹ vgs/leg at a constant volume of 20 μl per leg (FIG. 2). Mock animals were administered with a similar volume of vehicle (PBS). Thirty-three weeks after vector administration, TA muscle samples were assessed for the persistent ability to express dystrophin at the sarcolemmal membrane by immunofluorescence. The muscle cryosections were stained for dystrophin glycoprotein complex (DGC) proteins (FIG. 3A). The CAG-Kozak-micro-dystrophin delivered by AAV9WT vectors showed continued localized expression of dystrophin, dystroglycan and dystrobrevin proteins at the membrane. A similar expression pattern was observed in the muscles administered with AAV9WT-MHCK7-micro-dystrophin. The dystrophin positive fibers were quantified to evaluate the therapeutic efficacy. The AAV9WT-μDys group had significantly higher mean dystrophin stained myofibres than the mock (22.76% vs 1.68%, p<0.0001) and AAV9WT-MHCK7-μDys groups (35.60% vs 22.76%, p<0.0001) (FIG. 3B). Thus, the study revealed the superior efficiency of the CAG-Kozak construct in driving sustained micro-dystrophin expression at a longer follow-up of 33 weeks. Therefore, CAG-Kozak-micro-dystrophin construct was employed for the subsequent experiments.

### Development and Validation of Engineered AAV9 Vectors In Vitro and In Vivo

Subsequently, the Inventors developed next generation AAV9 vectors by engineering the capsids at PTM sites and packaged them with pssAAV-CAG-Kozak-μDys transgene construct to generate AAV9WT-CAG-Kozak-μDys (AAV9WT-μDys), AAV9K51Q-CAG-Kozak-μDys (AAV9K51Q-μDys) and AAV9N57Q-CAG-Kozak-μDys (AAV9N57Q-μDys) (Table 1). The engineered AAV9 vectors were assessed for their transduction efficiency in HeLa and C2C12 cells. Gene expression analysis for μDys revealed that AAV9K51Q-μDys had 1.33-fold (p≤0.05) higher expression when compared to AAV9WT-μDys whereas AAV9N57Q-μDys showed 2.94-fold (p<0.01) increased expression of μDys when compared to AAV9WT-μDys in Hela cells (FIG. 4A). In C2C12 cells, a significantly increased levels of μDys was observed in both AAV9K51Q-μDys (268-fold, p≤0.001) and AAV9N57Q-μDys (96-fold, p≤0.001) cells when compared to AAV9WT-μDys transduced cells (FIG. 4B). These in vitro transduction assays confirm that the AAV9WT and engineered vectors AAV9 are functional upon infection and are capable of expressing the μDys transgene.

a. The Inventors further evaluated the efficiency of engineered AAV9 vectors in vivo by intramuscular vector administration. Four groups of animals, (n=8 per group) were mock-administered or administered with AAV9WT-μDys, AAV9K51Q-μDys and AAV9N57Q-μDys vectors at a dose of 2×10¹¹ vgs/leg at a constant volume. The phenotypic rescue was studied 12 weeks after vector administration by measuring the total muscle grip strength of mice by using a non-invasive grip strength meter. The grip strength in the hind limbs injected with engineered AAV9 vectors was found to be significantly higher when compared to the mock treated mice. A significant increase in hind limbs grip strength was also observed in AAV9K51Q-μDys vector treated group compared to the group treated with the AAV9WT-μDys (Mean force 0.73 Newton (N) and 0.55N respectively, p≤0.05) (FIG. 5A). A similar trend was observed for all the four limbs grip strength (FIG. 5B). A longer follow up (18 weeks) demonstrated the superior grip strength in both the engineered AAV9 vector treated groups when compared to AAV9WT-μDys treated mice (FIGS. 5C and 5D). This demonstrates that phenotypic rescue exerted by the AAV9K51Q-μDys vectors was stable and consistent when compared to that of muscle force generated by AAV9WT-μDys or AAV9N57Q-μDys vector administered mice.

b. To confirm that the phenotypic rescue is due to restoration of dystrophin expression, the Inventors performed immunostaining for DGC complex proteins in TA muscles. The vector administered animals were sacrificed, 24 weeks post treatment and the TA muscles were analyzed by immunofluorescence. The dystrophin and DGC complex protein expression was higher in AAV9K51Q-μDys and AAV9N57Q-μDys mutant vector administered group than AAV9WT-μDys treated mice (FIG. 6A). These data suggest that the bioengineered AAV9 vectors leads to a stable and prolonged expression of dystrophin and its proper localization in the membrane facilitating the assembly of other DGC proteins. Further, quantification of dystrophin positive myofibers in the TA muscle sections (n≥3 TA muscles and n≥3 sections per muscle) revealed a significant increase in dystrophin positive fibers in AAV9K51Q-μDys vector group when compared to mock (Mean: 58.83% vs 1.12%, p<0.0001) and AAV9WT-μDys (58.83% vs 36.14%, p<0.0001) vector treated cohort of mice (FIG. 6B). AAV9N57Q-μDys vector treated mice also demonstrated higher dystrophin positive fibers when compared to mock treated (45.40% vs 1.12%, p<0.0001) or AAV9WT-μDys vector treated mice (45.40% vs 36.14%, p<0.0001) (FIG. 6B).Considering the consistent performance of AAV9K51Q-μDys vectors in robust dystrophin expression in vitro, and in mitigating the phenotype in mdx mice, we utilized this capsid for further studies.

### Codon Optimized μDys Transgene Demonstrated Superior Efficacy In Vitro and In Vivo

The Inventors reasoned that codon optimization of the μDys cDNA sequence for human skeletal tissues may improve the translational efficiency and the transgene expression. Further, the Inventors constructed pssAAV-CAG-Kozak-CouDys (CAG-Koz-CouDys) by incorporating the chemical sysnthesized CouDys sequence within the pssAAV-CAG-backbone plasmid. This CAG-Koz-CouDys construct was then packaged in the AAV9K51Q capsid to produce AAV9K51Q-CAG-Kozak-CouDys vectors (AAV9K51Q-CouDys) (Table 1). Improved translational efficiency of vector carrying the codon optimised transgene was noted with the vectors in C2C12 cells. An immunocytochemical analysis revealed 1.3 fold (p<0.05) higher micro-dystrophin expression in the cells infected with AAV9K51Q-CouDys vector when compared to cells transduced with AAV9K51Q-μDys vector (FIGS. 7A and 7B).

The Inventors further evaluated the AAV9K51Q-CouDys vector in mdx mice by intramuscular administration of the vectors at the dose of 2×10¹¹ vgs/leg in TA muscles (FIG. 2). Three groups of mice (n=10/cohort) were used including AAV9K51Q-μDys, AAV9K51Q-CouDys and the control group administered with PBS. The phenotypic response, was assessed 8 weeks later, by measuring the grip strength of hind limbs and all four limbs. Hind limb grip strength of AAV9K51Q-μDys vector and AAV9K51Q-CouDys treated mice was found to be significantly higher when compared mock treated animals (Mean: 0.68N vs 0.28N and Mean 0.89N vs 0.28N, p≤0.0001, respectively) (FIG. 8A). Hind limbs grip strength of AAV9K51Q-CoμDys vector administered mice were significantly higher than that of AAV9K51Q-μDys vector treated mice (Mean: 0.89N vs 0.68N, p≤0.0001) (FIG. 8A). A similar finding was observed for all of the four limbs, wherein the four limbs grip strength of AAV9K51Q-CouDys was significantly high when compared to AAV9K51Q-μDys vector treated mice (Mean: 1.61N vs 1.35N, p≤0.05) (FIG. 8B). Further follow-up at the 12 and 16 weeks time point after muscle gene transfer showed a similar pattern of higher grip strength. (FIG. 8C-8F). These data confirm that AAV9K51Q-CouDys vector has a robust and prolonged impact in improving the muscle strength of DMD mice.

a. Subsequently, immunostaining of TA muscles from vector treated mice 4 months after gene therapy, revealed that the expression of dystrophin and other DGC proteins like dystrobrevin and dystroglycan was higher in the vector treated groups and that these proteins were properly localised to the sarcolemma (FIG. 9A). Additionally, the analysis of whole muscle section of dystrophin-stained muscles revealed a uniform dystrophin expression along the entire section (FIG. 10). The Inventors further observed that AAV9K51Q-CouDys treated TA muscles had significantly higher number of dystrophin positive fibres (Mean: 64.57% vs 52.67%, p<0.001) (FIG. 9B) when compared to animals that received the AAV9K51Q-μDys vector. We further observed that AAV9K51Q-CouDys treated TA muscles had significantly higher percentage of dystrophin-positive fibers (Mean: 64.57% vs 52.67%, p<0.001) (FIG. 9B) when compared to animals that received the AAV9K51Q-μDys vector. Additionally, Masson trichrome (MT) staining of TA muscle sections from treated mice showed a visible decrease in the fibrosis (Mock vs AAV9K51Q-μDys: 1.29 fold, p<0.0001; Mock vs AAV9K51Q-CouDys: 1.62 fold, p<0.0001), 4 months after intramuscular gene therapy (FIGS. 11A and 11B). Similarly, TA muscles from AAV9K51Q-CouDys had significantly lesser collagen deposition (˜1.25 fold, p<0.0001) than animals administered with AAV9K51Q-μDys vectors (FIG. 11B). Hematoxylin and Eosin (H & E) staining of TA muscles from AAV9K51Q vector treated mice had significantly reduced the number of centrally nucleated myofibers compared to mock treated mice. Specifically, mice administered with AAV9K51Q-CouDys demonstrated ˜2 fold lesser centrally nucleated myofibers than the mice treated with AAV9K51Q-μDys (Mean: 16.21% vs 35.87%; p<0.0001) signifying the rescue of skeletal muscle characteristic features in AAV9K51Q-CouDys vector treated mice (FIGS. 12A and 12B). Vector genome (vg) quantification was performed to detect the AAV-specific genome in the vector-treated muscles using qPCR. Our data reveals that AAV9K51Q-CouDys had significantly higher vg copies when compared to AAV9K51Q-μDys (Mean: 99750 vs 54293 vector genome copies per 20 ng of DNA) (FIG. 13). Taken together, our data demonstrates the superiority of AAV9K51Q-CouDys vector in improving dystrophin levels and the phenotypic rescue, up to 4 months after intramuscular gene therapy.

### MATERIALS AND METHODS

### Cell Lines and Reagents

Human cervical carcinoma cells (HeLa) were obtained from American Type Culture Collection (ATCC, Manassas, VA, United States). C2C12 murine myoblast cell line was purchased from National Center for Cell Science (NCCS, Pune, India). H9C2 was a kind gift from Dr. Ashok Kumar, IIT Kanpur.AAV-293 cell line was purchased from Stratagene (San Diego, CA, USA). The cells were grown in Iscove's modified Dulbecco's medium (IMDM; Gibco, Life Technologies, Carlsbad, CA, USA) supplemented with 10% bovine serum (Gibco) at 37° C. with 5% CO₂. The media also contained 10 mg/mL of the antibiotics-ciprofloxacin (HiMedia Laboratories, Mumbai, India) and piperacillin (MP Biomedicals, Santa Ana, CA, USA). The SYBR green qPCR master mix was procured from Promega (Madison, WI, USA).

The control plasmid pssAAV-MHCK7-μDys (a kind gift from Dr. Jeffrey Chamberlain, University of Washington) contains human microdystrophin (R4-R23/Δ71-78) under the control of muscle specific MHCK7 promoter. The donor plasmid (pLV-hsa-μDys/eGFP) was purchased from addgene (Plasmid #26810), which contains the same human microdystrophin (R4-R23/Δ71-78) transgene with an eGFP tag. This plasmid was used to synthesize pssAAV-CAG-Kozak-μDys (GenScript, Piscataway, NJ, USA). Further a codon optimized version of human microdystrophin (pssAAV-CAG-Kozak-CouDys), was generated using an internal algorithm from GenScript. The AAV9 capsid mutants were generated in AAV9WT rep/cap plasmids and were chemically sysnthesized (GenScript).

### Designing of Microdystrophin Plasmid Vectors

The truncated version of human dystrophin i.e, microdystrophin from donor plasmid (pLV-hsa-μDys) was restricted and ligated in recipient plasmid by replacing eGFP sequence downstream of CAG-Kozak sequence between the inverted terminal repeats of the AAV back bone plasmid to generate the pssAAV-CAG-Kozak-μDys (CAG-Koz-μDys) plasmid. The plasmids were synthesised (GenScript, Piscataway, NJ, USA) and the cloning was confirmed by Sanger sequencing and restriction digestion analysis. pssAAVMHCK7-μDys (MHCK7-μDys) (a kind gift from Dr. Jeffrey Chamberlain, University of Washington) was used as the control. Further the μDys sequence in the pssAAV-CAG-Kozak-μDys was codon optimized for human muscle tissue and codon optimised μDys (CouDys) was cloned into donor plasmid by replacing μDys sequence to generate pssAAV-CAG-Kozak-CouDys (GenScript)

### Generation of Mutant AAV9 Capsid Vectors

Protein sequence for AAV9-VP1 (GenBank: AAS99264.1) was retrived and used as the reference. An online bioinformatics program called NeddyPreddy was employed to identify putative Neddylation sites. The medium and high threshold levels were established using the tool's output. An elevated cutoff score of 0.92 out of 1 led to the selection of the AAV9K51 target. Additionally, the glycosylation mutant AAV9 was produced by utilizing the HexNAcylation (Glycosylation) site N57 in VP1, which had been previously identified in our study⁵². Using specific primers and the QuikChange II XL Site-Directed Mutagenesis Kit (Agilent Technologies, Santa Clara, CA, USA), a site-directed mutagenesis (SDM) of the AAV9WT rep/cap plasmid (pAAVR2/C9 WT) was carried out to replace the Neddylation site (K51) and glycosylation site (N57) in the AAV9 capsid with glutamine residue (Q). The final constructs were confirmed by Sanger sequencing.

### Production of Recombinant AAV Vectors

For AAV vector production, polyethyleneimine (PEI;Polysciences, Warrington, PA, USA)-based triple transfection of AAV 293 cells with equimolar concentration of mutant rep/cap plasmid (pAAVR2/C9 WT; pAAVR2/C9 K51Q; pAAVR2/C9 N517Q), transgene containing CAG-Koz-μDys or MHCK7-μDys or CAG-Koz-Co μDys along with adenoviral helper plasmid was performed. After a period of 72 hours, the transfected cells were collected, processed, and then the virus purified and concentrated as previously described. Using this protocol, the Inventors generated AAV9WT-CAG-Koz-μdys (AAV9WT-μdys); AAV9WT-MHCK7-μdys (AAV9WT-MHCK7-μdys), AAV9K51Q-CAG-Koz-μdys (AAV9K51Q-μdys), AAV9N57Q-CAG-Koz-μdys (AAV9N57Q-μdys) and AAV9K51Q-CouDys vectors. Virus quantification was done by quantitative (q) PCR with polyadenylation (PolyA) signal-specific primers (CFX96, Bio-Rad, Hercules, CA, USA).

### Transfection Studies

The CAG-Koz-μDys plasmid and MHCK7-μDys plasmid were transfected (500 ng) in HeLa, C2C12 and H9C2 cell lines using PEI. After 48 hours, the cells were collected and RNA was isolated using TRIzol (Invitrogen, MA, USA) and cDNA was prepared from 1 μg of RNA using Quantitect reverse transcription kit (Qiagen, Hilden, Germany). The levels of μDys were measured by qPCR using specific primers (Forward primer (FP): AACAAAGTGCCCTACTA; Reverse primer (RP): AGGTTGTGCTGGTCCA). 18srRNA (FP: TTGACGGAAGGGCACCACCAG; RP: GCACCACCACCCACGGAATCG) was used as reference gene for normalization of qPCR data.

### Transduction Assays

The transduction efficiency of the packaged AAV9WT-μDys, AAV9WT-MHCK7-μDys, AAV9K51Q-μDys, AAV9N57Q-μDys and AAV9K51Q-CouDys-μDys was assessed in vitro. Briefly, the HeLa, C2C12 and H9C2 cells were infected with the vectors at 1×10⁵ vgs MOI (multiplicity of infection). After 48 hours, the RNA from cells isolated and the μDys levels measured by qPCR as described above.

Immunocytochemistry was performed to validate the increased translation efficiency of AAV9K51Q vector packaged with optimized transgene. Briefly C2C12 cells were seeded at a density of 10⁵ cells per well over the coverslip precoated with Poly-L-Lysine in 6 well format. Cells were then infected with AAV9K51Q-μDys/AAV9K51Q-CouDys at an MOI of 1×10⁵ vgs and mock cells were treated with IMDM. After 24 hours of transduction, the cells were fixed in 4% paraformaldehyde. Further post blocking, the cells were incubated with primary antibody for Dystrophin (1:150, sc-33697 Santa Cruz, Dallas, TX, USA) for 1 hour followed by washing with 1×PBS, thrice. The cells were then stained with secondary antibody goat anti-mouse Alexa-fluor 568 (1:250, Thermofisher) for 1 hour followed by washing with 1×PBS thrice. Cells were then counterstained with DAPI (1:1000 v/v, Sigma Aldrich) followed with mounting the coverslip over the glass slide with Flurosave (Sigma Aldrich). The images were obtained in ZOE-Fluorescent cell imager (Biorad, Hercules, CA, USA). The images (n≤8/group) were used for semi-quantification analysis using ImageJ software.

### Intramuscular Gene Therapy in Mdx Mice

DMD mice (B6Ros.Cg-Dmd^{mdx-4cv}/J) (mdx) were purchased from Jackson Laboratory (Bar Harbor, ME). The animal experiments were approved by the IIT-Kanpur Institutional Animal Ethics Committee (IAEC). For vector administration, 6-10 weeks old mice were used. For the initial studies to identify the optimum μDys transgene cassettes, 3 groups of mice, namely, Mock (n=8), AAV9WT-MHCK7-μDys (n=8) and AAV9WT-μDys (n=8) were utilized. The experimental mice were adminstered with the respective vectors at the dose of 3.42×10¹¹ vgs/leg with constant volume of 20 μl in their TA muscle (FIG. 2). Mock group animals were administered with same volume of 1×PBS. In the second batch of in vivo experiments to study the efficacy of bioengineered AAV9 vectors, four groups-Mock (n=8), AAV9WT-μDys (n=8), AAV9K51Q-μDys (n=7) and AAV9N57Q-μDys (n=8) of mice were injected with the vectors at 2×10¹¹ vgs/leg. Further, in the last set of experiments to characterize the efficacy of CouDys, animals were grouped into mock-treated (n=10) or administered with AAV9K51Q-μDys (n=10) and AAV9K51Q-CouDys (n=9) vectors (2×10¹¹ vgs/leg) in their TA muscle.

### Muscle Strength Analysis

The muscular strength of hind limbs of mock-treated and vector treated DMD mice was measured using grip strength meter (Bioseb, GS-4, Chaville, France). Briefly, animals were scuffed and were allowed to grab the mesh wire with their fore limbs, and the hind limbs of the mice was allowed to grasp the T rod attached to the grip strength meter. Once the mice grabbed the T rod using hind limbs, the animal was pulled gently using the tail until hind limbs were released from the T rod and the peak grip strength force in newtons was recorded. Similarly, to assess the grip strength for all the four limbs, mice was placed on the grid attached to grip strength meter, after it grasped the grid with all its four limbs, the animal was pulled gently using its tail until it released the grid. For each mouse, five peak grip strength readings were taken and the average was computed. The muscle grip strength in vector treated mice were assessed at different time points, ranging from 8-24 weeks after vector administration.

### Immunohistochemistry

Animals were euthanized, at different time points (17, 24 and 33 post vector administration) and TA muscles were excised as described earlier. After removing the excess moisture from the muscles, snap-freezing was performed. The muscles were first covered by a layer of OCT and then frozen in isopentane cooled in liquid nitrogen, for 60 seconds. The muscles were then kept in dry ice for few minutes to remove the isopentane followed by transfer in moulds and covered with OCT and kept in dry ice until it was frozen. After freezing, the moulds were stored at −80° C. until sectioning. The muscle tissues were sectioned using cryotome (Leica Biosystems, Wetzlar, Germany). Muscle sections of 8 μm thickness were obtained and transferred to Poly-L-Lysine coated Superfrost slides (VWR, Radnor, PA) and stored at −20° C. until staining. For staining, the sections were fixed with chilled solution of 4% paraformaldehyde. After blocking, the sections were incubated with primary antibodies for Dystrophin (1:50), β-Dystroglycan (1:50, 1610381, Biorad) and Dystrobrevin (1:50, 610766, BD Bioscience, NJ, USA) for 24 hours at 4° C. followed by washing three times with 1×PBS for 10 minutes each. The sections were then incubated with secondary antibody goat anti-mouse Alexa-fluor 568 (1:250, Thermofisher) for 24 hours at 4° C. followed by washing three times with 1×PBS for 10 minutes each. Counterstaining of sections was done using DAPI (1:1000 v/v) and mounted using fluorsave. The images were obtained in confocal microscope at 40× magnification (Advanced High Sensitive Spectral Confocal and Multiphoton Microscope LSM780NLO, CarlZeiss GmbH, Wein, Austria and AXR Nikon microscope-High-end confocal). To quantify the total fibers, muscle sections were stained with Alexa fluor 568-tagged wheat germ agglutinin (WGA) (W56133, Thermofisher). Briefly, fixed sections were incubated with WGA (5 μg/mL) for 2 hrs. The sections were washed in 1×PBS and counter-stained with DAPI followed by mounting. The sections were then imaged in 20× magnification with an inverted microscope (Dmi8, Leica Microsystems, Germany), to quantify the total fiber number as well as dystrophin-positive fibers. Images were acquired from three different animals, with three independent sections per animal, to determine total fiber count from WGA-stained sections and quantify dystrophin-positive fibers from dystrophin-stained images. The quantification was performed using the multipoint tool in Image J as previously described.

### Morphometric Assessment in Vector-Treated Mice

Hematoxylin and Eosin (HE) staining and Masson trichrome (MT) staining of TA muscle section (8 μm thickness) was performed using standard protocols to assess the muscle morphological features and fibrosis respectively. Images were captured in an inverted microscope (Dmi8, Leica Microsystems, Germany). Centrally nucleated myofibers were counted manually using multi pint tool in ImageJ software (n≥15 images/group and n≥2 tissues/group). MT staining images were used for the measurement of fibrotic areas (n≥10 images/group and n=3 tissues/group) using the color devolution plugin in Image J software. The original images were segmented into three clusters. The blue areas of the image represent the collagen deposition whose total area was measured by correcting the threshold manually such that the collagen deposition overlays with the original histology image.

### Vector Genome Quantification

Total genomic DNA was isolated from the vector-administered TA muscles (n=4 per group). Vector genome copy numbers in 20 ng of DNA was determined by a quantitative PCR using ITR-specific primers (Forward primer: GGAACCCCTAGTGATGGAGTT, Reverse primer: CGGCCTCAGTGAGCGA) as described earlier.

### Statistical Analysis

Data are presented as mean±standard deviation (SD). qPCR data was analyzed by the Biorad CFX Manager 3.1. Statistical significance was evaluated using one way ANOVA, executed with GraphPad Prism 8.0.2 software.

We claim:

1. A bioengineered AAV9 vector comprising Optimized Transgene for Duchenne muscular dystrophy gene therapy comprising a novel mutation present at the post translational site/s of the vector;
said gene sequence comprises optimised microdystrophin transgene with ubiquitous promoter comprising CAG promoter and Kozak sequence upstream of the transgene.

2. The bioengineered AAV9 vector as claimed in claim 1, comprises AAV9 and its mutant vectors comprising K51Q, N57Q, comprising sequence Id no. 2 and 3 respectively.

3. The bioengineered AAV9 vector as claimed in claim 1, wherein ubiquitous promoter containing micro-dystrophin vector is constructed by the steps of:
constructing the plasmid vector by sub-cloning the ΔR4-23/ΔC micro-dystrophin gene (μDys) from the donor plasmid pLV-I-μDys to the recipient plasmid pssAAV-CAG-eGFP, wherein the recipient plasmid comprises a hybrid promoter/enhancer sequence with the combination of a cytomegalovirus (CMV) immediate early enhancer sequence and chicken β actin (CBA) promoter; and
inserting a consensus ribosome binding site sequence comprising Kozak sequence being inserted downstream to the CAG promoter, such that the micro-dystrophin gene is placed downstream to the CAG-Kozak sequence in the final construct of pssAAV-CAG-Kozak-micro-dystrophin (CAG-Kozak-μDys) comprising sequence Id 5, to ensure the ubiquitous and greater translational efficacy of dystrophin.

4. The bioengineered AAV9 vector as claimed in claim 1, wherein the truncated version of human dystrophin comprising microdystrophin from donor plasmid (pLV-hsa-μDys) is restricted and ligated in recipient plasmid by replacing eGFP sequence downstream of CAG-Kozak sequence between the inverted terminal repeats of the AAV back bone plasmid to obtain the pssAAV-CAG-Kozak-μDys (CAG-Koz-μDys) microdystrophin plasmid.

5. The bioengineered AAV9 vector as claimed in claim 1, wherein next generation AAV9 vectors is constructed by engineering the capsids at PTM sites and packaging them with pssAAV-CAG-Kozak-μDys transgene construct to obtain AAV9WT-CAG-Kozak-μDys (AAV9WT-μDys), AAV9K51Q-CAG-Kozak-μDys (AAV9K51Q-μDys) and AAV9N57Q-CAG-Kozak-μDys (AAV9N57Q-μDys).

6. The bioengineered AAV9 vector as claimed in claim 1, wherein the AAV9 based vector is injected by intramuscular or systemic administration at the dose range of 1×10¹⁰ vgs to ×10¹⁴ vgs.

7. A method of preparing a bioengineered AAV9 vector carrying optimized transgene for Duchenne muscular dystrophy gene therapy comprising the steps of:
selection of a hybrid chicken β-actin promoter and cytomegalovirus early enhancer (CAG) for expression of dystrophin;
incorporating a consensus ribosome binding site sequence or Kozak sequence, after the CAG promoter upstream of the transgene sequence to ensure increased and robust expression of the therapeutic protein;
performing capsid engineering at PTM sites to generate AAV9 mutant vectors with increased transduction efficiency; and
performing codon optimization of the μDys (CouDys) transgene for optimal expression in human skeletal muscles.

8. The method as claimed in claim 6, wherein codon optimization of the μDys (CouDys) transgene is performed by the steps:
i.constructing pssAAV-CAG-Kozak-CouDys (CAG-Koz-CouDys) comprising sequence Id 6, by incorporating the chemically synthesized CouDys sequence within the pssAAV-CAG-backbone plasmid; and
ii.packaging CAG-Koz-CouDys construct in AAV9K51Q capsid to obtain AAV9K51Q-CAG-Kozak-CouDys vectors (AAV9K51Q-CouDys).

9. The method as claimed in claim 7, wherein the AAV9 based vector is injected by intramuscular or systemic administration at the dose range of 1×10¹⁰ vgs to ×10¹⁴ vgs.
